(19) United States
(12) Patent Application Publication
(10) Pub. No.: US 2009/0137025 A1
(43) Pub. Date: May 28, 2009

(54) Apparatus for containing, cultivating, and harvesting photosynthetic marine microorganisms within water

(75) Inventors: James Stephens, Snohomish, WA (US); Kirk W. Dickinson, Newcastle, WA (US); Kelly Ogilvie, Seattle, WA (US)

Correspondence Address: LAW OFFICES OF MICHAEL DRYJA, 1474 N COOPER RD #105-248, GILBERT, AZ, 85233, US

(73) Assignee: GREEN VISION ENERGY CORPORATION, Seattle, WA (US)

(21) Appl. No.: 11/944,610

(22) Filed: Nov. 24, 2007

(51) Int. Cl. C12M 1/36; C12R 1/89

(52) U.S. Cl. 435/286.2

(57) ABSTRACT

An apparatus contains photosynthetic marine microorganisms within water, to permit the marine microorganisms to be cultivated and harvested. A self-supporting buoyant frame of the apparatus is to be placed within the water. A mesh lining of the apparatus is situated within the interior of the frame. The mesh lining is adapted to permit the water and nutrients to enter through the mesh lining while at least substantially preventing the marine microorganisms that are being cultivated from escaping through the mesh lining. A funnel of the apparatus is attached to a corresponding hole at a bottom of the mesh lining, and is adapted to permit the marine microorganisms that have been cultivated to be harvested. A buoyancy leveling subsystem of the apparatus is adapted to control an extent to which the frame is submerged within the water.

### RELATED PATENT APPLICATIONS

The present patent application is related to the cofiled, copending, and coassigned patent application entitled “method for removing undesirable components from water while containing, cultivating, and harvesting photosynthetic marine microorganisms within water,” the contents of which are hereby incorporated in their entirety by reference.

### BACKGROUND

Photosynthetic marine microorganisms include micro- and macro-sized algae, among other types of such microorganisms. While photosynthetic marine microorganisms grow naturally, cultivating them purposefully in large numbers has become attractive due to the increasing value of these microorganisms. For example, algae in particular has become for bio-fuel production, energy generation, agricultural material production, and carbon sequestration purposes, among other purposes. However, efficiently and inexpensively intentionally growing photosynthetic marine microorganisms like algae has proven relatively difficult.

### SUMMARY

The present invention relates to an apparatus for containing, cultivating, and harvesting photosynthetic marine microorganisms, such as algae, within water. Such an apparatus of one embodiment of the invention includes a self-supporting buoyant frame, a mesh lining, a funnel, and a buoyancy leveling subsystem. The frame is to be placed within the water. The mesh lining is situated within the interior of the frame, and is adapted to permit the water and nutrients to enter through the mesh lining while at least substantially preventing the marine microorganisms that are being cultivated from escaping through the mesh lining. The funnel is attached to a corresponding hole at the bottom of the mesh lining, and is adapted to permit the marine microorganisms that have been cultivated to be harvested. The buoyancy leveling subsystem is adapted to control the extent to which the frame is submerged within the water.

In one embodiment of the invention, the frame may be fabricated from hollow tubing. The hollow tubing has an interior space that is receptive to at least the water and a gas, such as air, in different combinations. In such an embodiment, the buoyancy leveling subsystem may include a first valve and a second valve. The first valve is disposed through the hollow tubing of the frame, and is located at or near a top of the frame. The second valve is also disposed through the hollow tubing of the frame, but is located at or near the bottom of the frame. The second valve may be one or more holes within the hollow tubing of the frame.

The first valve has a number of mutually exclusive states in which the first valve is adapted to operate. A first such state is an open state in which the first valve externally exposes the interior space of the hollow tubing of the frame, such that the level of the water within the hollow tubing of the frame rises and the extent to which the frame is submerged within the water increases. A second such state is a closed state in which the first valve at least substantially does not externally expose the interior space of the hollow tubing of the frame, such that the level of the water within the hollow tubing of the frame remains substantially constant and the extent to which the frame is submerged within the water remains substantially constant. A third such state is a gas-transfer state in which gas is pumped into the hollow tubing through the first valve, such that the level of the water within the hollow tubing of the frame lowers, and the extent to which the frame is submerged within the water decreases.

Embodiments of the invention provide for advantages over the prior art. The apparatus for containing, cultivating, and harvesting photosynthetic marine microorganisms, such as algae, within water is inexpensively manufactured and efficiently utilized. A number of such apparatuses can be placed in a body of water for growing algae, where the apparatuses can be controlled by their buoyancy leveling subsystems so that they are substantially submerged within the water during cultivation of the marine microorganisms. The mesh linings ensure that the microorganisms being grown do not escape the apparatuses. Once the marine microorganisms have been sufficiently cultivated, they can be harvested by first raising the apparatuses within the water through use of the buoyancy leveling subsystems, and then transferring the microorganisms via the funnels attached to the mesh linings.

Still other aspects, advantages, and embodiments of the invention will become apparent by reading the detailed description that follows, and by referring to the accompanying drawings.

### BRIEF DESCRIPTION OF THE DRAWINGS

The drawings referenced herein form a part of the specification. Features shown in the drawing are meant as illustrative of only some embodiments of the invention, and not of all embodiments of the invention, unless otherwise explicitly indicated, and implications to the contrary are otherwise not to be made.

FIG. 1 is a diagram of a top view of an apparatus for containing, cultivating, and harvesting photosynthetic marine microorganisms within water, according to an embodiment of the invention.

FIG. 2 is a diagram of a front view of an apparatus of FIG. 1, according to an embodiment of the invention.

FIG. 3 is a diagram depicting the apparatus of FIGS. 1 and 2 in which the apparatus is substantially submerged within water for cultivation of photosynthetic marine microorganisms, according to an embodiment of the invention.

FIG. 4 is a diagram depicting the apparatus of FIGS. 1 and 2 in which the apparatus is substantially raised above the water for harvesting the photosynthetic marine microorganisms that have been cultivated, according to an embodiment of the invention.

### DETAILED DESCRIPTION OF THE DRAWINGS

In the following detailed description of exemplary embodiments of the invention, reference is made to the accompanying drawings that form a part hereof, and in which is shown by way of illustration specific exemplary embodiments in which the invention may be practiced. These embodiments are described in sufficient detail to enable those skilled in the art to practice the invention. Other embodiments may be utilized, and logical, mechanical, and other changes may be made without departing from the spirit or scope of the present invention. The following detailed description is, therefore, not to be taken in a limiting sense, and the scope of the present invention is defined only by the appended claims.

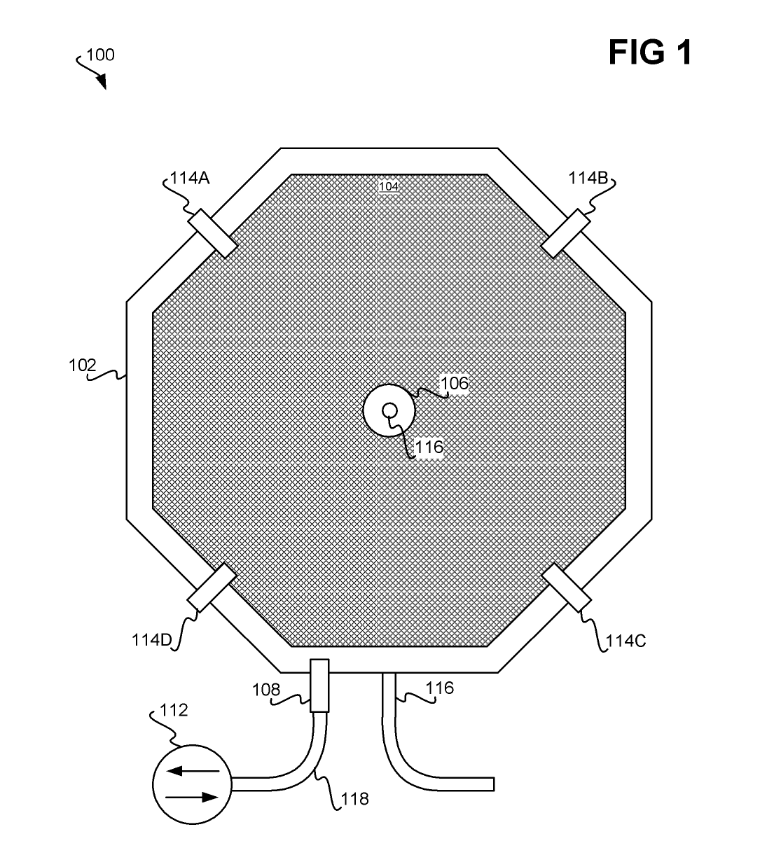

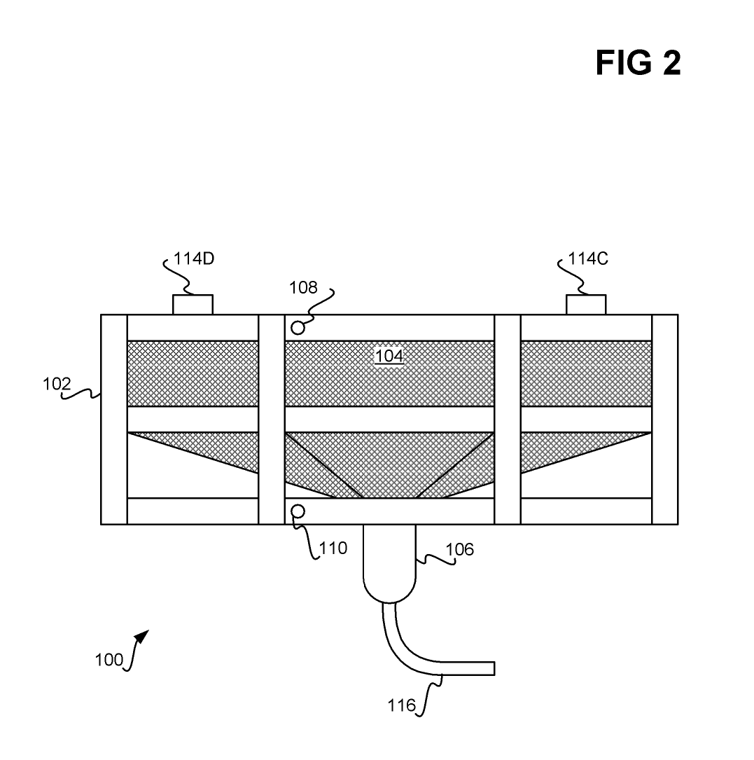

FIGS. 1 and 2 show a top view and a front view, respectively, of an apparatus 100 for containing, cultivating, and harvesting photosynthetic marine microorganisms, such as micro- and macro-sized algae, within water, according to an embodiment of the invention. The apparatus 100 includes a self-supporting buoyant frame 102. The frame 102 is self-supporting in that it does not require any additional components or members to support it. The frame 102 is buoyant in that it can float in water. The frame 102 may have an octagonal shape, as is specifically depicted in FIGS. 1 and 2, or it may have a different shape, such as a circular, square, rectangular, oval, and/or prismatic shape, among other types of shapes.

The frame 102 is fabricated from a durable material such that the frame 102 has sufficient structural strength and is also buoyant. For example, the frame 102 may be fabricated from hollowing tubing. The hollow tubing has an interior space that is receptive to water and gas, such as air, in different combinations. In one embodiment, the frame 102 may be free-floating.

Additionally, or alternatively, the apparatus 100 may include a number of anchoring points 114A, 114B, 114C, and 114D, collectively referred to as the anchoring points 114. While there are four anchoring points 114 in FIGS. 1 and 2, there may be more or less of such anchoring points 114 in other embodiments. The anchoring points 114 permit the frame 102 to be floatatively anchored in place, to be moved within the water, as well as to be lifted from the water, such as by employing a crane.

The apparatus 100 includes a mesh lining 104 within the interior of the frame 102. The mesh lining 104 thus defines a space within the apparatus 100. The mesh lining 104 is adapted to permit water and the nutrients needed for the marine microorganisms to grow to enter the space through the mesh lining 104, while at least substantially preventing the marine microorganisms from escaping the space within which they are being cultivated through the mesh lining 104. The mesh lining 104 may be fabricated from metal and/or fabric in one embodiment.

The mesh lining 104 specifically is or has a micron-sized mesh in one embodiment. Through experimentation, the inventors have determined that a mesh of one to forty microns in size is preferred to permit nutrients to enter through the mesh lining 104 while at least substantially preventing the marine microorganisms from escaping through the mesh lining 104. A mesh of one to forty microns in size means that the openings defined by the mesh are each one to forty microns in size.

The apparatus 100 includes a funnel 106, such as a cone, attached to a corresponding hole at the bottom of the mesh lining 104, substantially in the center of the mesh lining 104 in one embodiment. The funnel 106 is adapted to permit the marine microorganisms that have been cultivated to be harvested. In particular, the funnel 106 has a first opening at which the funnel 106 is attached to the corresponding hole of the mesh lining 104, and a second opening at which a hose 116 is removably connected. The first opening may be larger in size than the second opening.

When the hose 116 is not attached to the funnel 106, the opening in question is capped or otherwise closed. When the hose 116 is attached to the funnel 106, a pump or another piece of equipment can be employed to suction the marine microorganisms that have been cultivated from the apparatus 100 for further processing. Removing the marine microorganisms from the apparatus 100 is thus what is meant by harvesting in this respect.

The apparatus 100 includes a buoyancy leveling subsystem that includes a top valve 108, a bottom valve 110, and/or a compressor 112 in one embodiment. The buoyancy leveling subsystem is generally adapted to control the extent to which the frame 102 is submerged within the water in which the frame 102 has been placed. For instance, while the marine microorganisms are being cultivated, the buoyancy leveling subsystem is controlled so that the majority of the frame 102 is submerged. By comparison, when the marine microorganisms are ready to be harvested, the buoyancy leveling subsystem is controlled so that the majority of the frame 102 is not submerged.

Both the valves 108 and 110 are disposed within the hollow tubing of the frame 102 such that they are fluidically connected with the interior space within the hollow tubing. The top valve 108 is located at or near the top of the frame 102, while the bottom valve 110 is located at or near the bottom of the frame 102. In one embodiment, the bottom valve 110 may specifically be simply one or more holes within the frame 102, where these holes remain open substantially all the time, externally exposing the interior space of the hollow tubing.

The top valve 108 in one embodiment is positioned on the frame 102 so that the valve 108 is never submerged underwater, regardless of the extent to which the frame 102 is submerged. Likewise, the bottom valve 110 in one embodiment is positioned on the frame 102 so that the valve 110 is always submerged underwater, regardless of the extent to which the frame 102 is submerged. In the embodiment where the bottom valve 110 always externally exposes the interior space of the hollow tubing of the frame 102, this means that the hollow tubing is always externally exposed to water while the apparatus 100 is being employed.

The top valve 108 has a number of mutually exclusive states in which it is adapted to operate. The top valve 108 is operated in different of these states to control the extent to which the frame 102 is submerged within the water. In an open state, the top valve 108 is opened to externally expose the interior space of the hollow tubing of the frame 102. In this state of the top valve 108, the level of the water within the hollow tubing of the frame 102 increases, and the extent to which the frame 102 is submerged within the water increases. This is because water enters the bottom valve 110, naturally displacing the gas, such as air, that had been in the hollow tubing and which had previously maintained the frame 102 at a higher level within the water.

In a closed state, the top valve 108 is closed to at least substantially not externally expose the interior space of the hollow tubing of the frame 102. In this state of the top valve 108, the level of the water within the hollow tubing of the frame 102 remains at least substantially constant, and the extent to which the frame 102 is submerged within the water remains at least substantially constant. This is because the water and the gas, such as air, within the hollow tubing remains at a substantially constant combination. The gas cannot escape from the top valve 110, so no water enters the bottom valve 110, even though it is open, because the water has nothing to displace.

In a gas-transfer state, the top valve 108 is fluidically and removably connected to the compressor 112 via a hose 118. The compressor 112 forcibly pumps gas, such as air, into the hollow tubing of the frame 102 through the top valve 108. In this state of the top valve 108, the level of the water within the hollow tubing of the frame 102 decreases, and the extent to which the frame 102 is submerged within the water decreases. This is because the gas pushes, or displaces, at least some of water from the hollow tubing through the bottom valve 110.

Therefore, when marine microorganisms are to be cultivated within the apparatus 100, the top valve 108 may be opened to enter the open state, so that the frame 102 sinks to a lower level within the water. Once the frame 102 has reached the desired (lower) submersion level, the top valve 108 is closed to enter the closed state, in which the frame 102 remains at this submersion level within the water. When the microorganisms are ready to be harvested, the top valve 108 is opened and the compressor 112 fluidically connected thereto via the hose 118 so that the valve 108 enters the gas-transfer state. The compressor 112 is turned on so that the frame 102 rises to a higher level within the water. Once the frame 102 has reached the desired (higher) submersion level, the top valve 108 is again closed to enter the closed state, and the frame 102 remains at this submersion level within the water so that the microorganisms can be harvested.

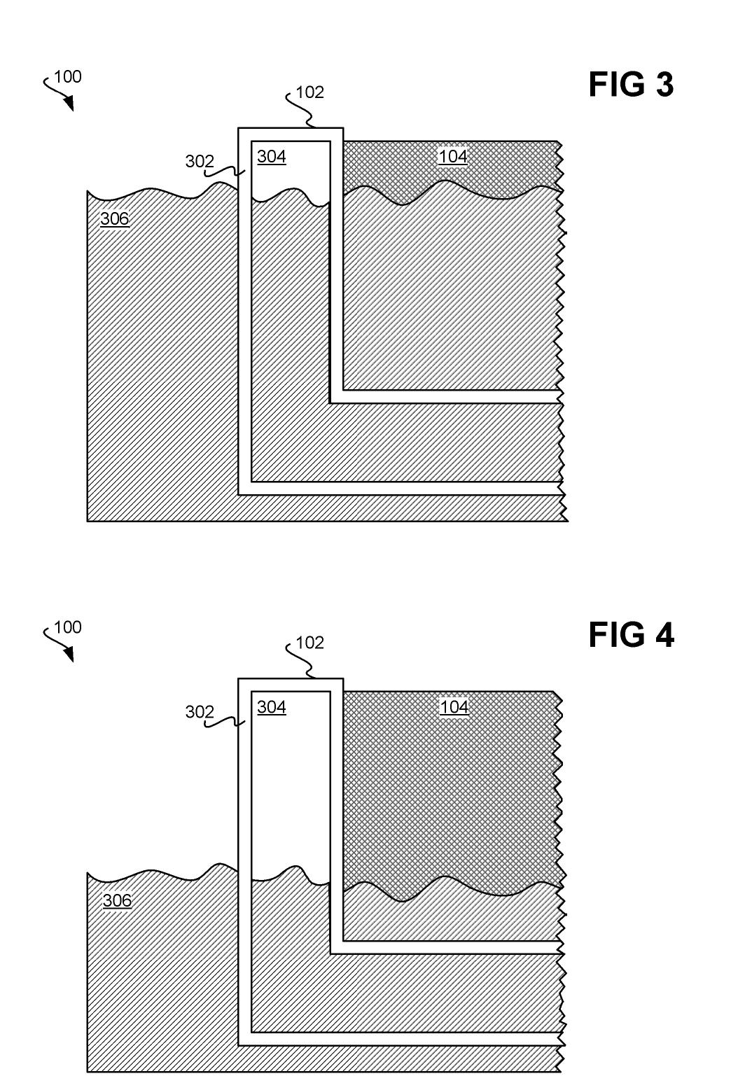

FIG. 3 shows a portion of the apparatus 100 when it is substantially submerged within water 306 for the cultivation of photosynthetic marine microorganisms like algae, according to an embodiment of the invention. The frame 102 is depicted as including hollow tubing 302. The top valve 108 (not depicted in FIG. 3) is opened so that the level of the water 306 within the interior space of the hollow tubing 302 rises due to intake of the water 306 through the bottom valve 110 (also not depicted in FIG. 3), resulting in the apparatus 100 sinking within the water 306 to a desired (lower) level.

The valve 108 is then closed so that the level of the water 306 within the interior space of the hollow tubing 302 remains constant. As such, a majority of the apparatus 100, including the frame 102 and the mesh lining 104, is submerged. Insofar as an upper portion of the apparatus 100 is not submerged within the water 306, an upper portion of the hollow tubing 302 includes gas 304, such as air, therewithin, instead of water 306.

In the position of FIG. 3, the apparatus 100 remediates nitrogen and phosphorous contamination from water via the growth of marine microorganisms like algae that photosynthetically feed on such contaminants. A limiting nutrient may be placed in the water 306 to control the rate at which the marine microorganisms grow. The mesh lining 104 ensures that the microorganisms do not escape from the apparatus 100, while ensuring that the water 306, nutrients, and so on, are able to reach the microorganisms. Time is allowed to pass with the apparatus 100 in the position of FIG. 3, until the marine microorganisms have grown to a desirable amount or concentration.

FIG. 4 shows a portion of the apparatus 100 when it has been substantially raised from the water 306 for the harvesting of photosynthetic marine microorganisms like algae that have already been cultivated, according to an embodiment of the invention. The frame 102 again includes the hollow tubing 302. The top valve 108 (not depicted in FIG. 4) is fluidically connected to the compressor 112 (also not depicted in FIG. 4) to increase the amount of gas 304, such as air, within the interior space of the hollow tubing 302. As such, the level of the water 306 within this interior space lowers due to the water 306 being forced out through the bottom valve 110 (also not depicted in FIG. 4), resulting in the apparatus 100 rising within the water 306.

The valve 108 is then closed so that the level of the water 306 within the interior space of the hollow tubing 302 remains constant. As such, substantially less than a majority of the apparatus 100, including the frame 102 and the mesh lining 104, is submerged. Insofar as a lower portion of the apparatus 100 is still submerged within the water 306, a lower portion of the hollow tubing 302 still includes the water 306.

In the position of FIG. 4, the marine microorganisms, such as algae, that have been previously cultivated in the position of FIG. 3 can be harvested. The apparatus 100 is substantially raised from the water 306, and in so doing substantially separating the marine microorganisms from the water 306 to result in a denser, more commercially viable microorganism slurry. This slurry can then be pumped from the bottom of the apparatus 100 via the hose 116 (not depicted in FIG. 4) onto a vessel, platform, or other location for further processing into refinable materials. Such refinable materials can include lipid oil for biodiesel and ethanol production, as well as biomass for methane generation, carbon sequestration, and fertilizer and/or animal feed production.

It is noted that, although specific embodiments have been illustrated and described herein, it will be appreciated by those of ordinary skill in the art that any arrangement calculated to achieve the same purpose may be substituted for the specific embodiments shown. This application is thus intended to cover any adaptations or variations of embodiments of the present invention. Therefore, it is manifestly intended that this invention be limited only by the claims and equivalents thereof.

We claim:

1. An apparatus for containing, cultivating, and harvesting photosynthetic marine microorganisms within water, comprising:
a self-supporting buoyant frame to be placed within the water;
a mesh lining within the interior of the frame, the mesh lining adapted to permit the water and nutrients to enter through the mesh lining while at least substantially preventing the marine microorganisms that are being cultivated from escaping through the mesh lining;
a funnel attached to a corresponding hole at a bottom of the mesh lining, the funnel adapted to permit the marine microorganisms that have been cultivated to be harvested; and,
a buoyancy leveling subsystem that is adapted to control an extent to which the frame is submerged within the water.

2. The apparatus of claim 1, further comprising one or more anchoring points adapted to one or more of:
permit the frame to be anchored in place;
permit the frame to be moved within the water; and,
permit the frame to be lifted from the water.

3. The apparatus of claim 1, wherein the frame is adapted to be free-floating.

4. The apparatus of claim 1, wherein the frame is fabricated from a durable material such that the frame has sufficient structural strength as well as buoyancy.

5. The apparatus of claim 1, wherein the frame has one or more of: an octagonal shape, a circular shape, a square shape, a rectangular shape, an oval shape, and a prismatic shape.

6. The apparatus of claim 1, wherein the frame is fabricated from hollow tubing, the hollow tubing having an interior space receptive to at least the water and gas in different combinations.

7. The apparatus of claim 6, wherein the buoyancy leveling subsystem comprises:
a first valve disposed through the hollow tubing of the frame and located at or near a top of the frame; and,
a second valve disposed through the hollow tubing of the frame and located at or near the bottom of the frame.

8. The apparatus of claim 7, wherein the first valve has a plurality of mutually exclusive states in which the first valve is adapted to operate, the mutually exclusive states essentially consisting of:
an open state in which the first valve externally exposes the interior space of the hollow tubing of the frame;
a closed state in which the first valve at least substantially does not externally expose the interior space of the hollow tubing of the frame; and,
a gas-transfer state in which gas is pumped into the hollow tubing through the first valve,
wherein the first valve is operated in different of the mutually exclusive states to control the extent to which the frame is submerged within the water.

9. The apparatus of claim 8, wherein the second valve is operated in a substantially permanent open state in which the second valve externally exposes the interior space of the hollow tubing of the frame,
such that while the first valve is being operated in the open state, a level of the water within the hollow tubing of the frame rises, and the extent to which the frame is submerged within the water increases,
such that while the first valve is being operated in the closed state, the level of the water within the hollow tubing of the frame remains substantially constant, and the extent to which the frame is submerged within the water remains substantially constant, and
such that while the first valve is being operated in the gas-transfer state, the level of the water within the hollow tubing of the frame lowers, and the extent to which the frame is submerged within the water decreases.

10. The apparatus of claim 7, wherein the second valve comprises one or more holes within the hollow tubing of the frame.

11. The apparatus of claim 7, wherein the buoyancy leveling subsystem further comprises a compressor removably connectable to the first valve to pump gas into the hollow tubing through the first valve.

12. The apparatus of claim 1, wherein the mesh lining has a micron-sized mesh.

13. The apparatus of claim 12, wherein the micron-sized mesh is from one to forty microns in size.

14. The apparatus of claim 1, wherein the mesh lining is fabricated from one or more of metal and fabric.

15. The apparatus of claim 1, wherein the mesh lining defines a space within which the marine microorganisms are cultivated.

16. The apparatus of claim 1, wherein:
the funnel defines a first opening at which the funnel is attached to the corresponding hole at the bottom of the mesh lining,
the funnel defines a second opening to which a hose is connected, the marine microorganisms that have been cultivated being removed from the mesh lining through the funnel and then through the hose, and
the first opening of the funnel is larger in size than the second opening of the funnel.

17. The apparatus of claim 16, further comprising the hose.

18. The apparatus of claim 1, wherein the marine microorganisms comprise macro-sized and micro-sized marine algae.

19. An apparatus for containing, cultivating, and harvesting photosynthetic marine microorganisms within water, comprising:
a self-supporting buoyant frame to be placed within the water, the frame fabricated from hollow tubing, the hollow tubing having an interior space receptive to at least the water and gas in different combinations;
a mesh lining within the interior of the frame and defining a space within which the marine microorganisms are to be cultivated, the mesh lining adapted to permit the water and nutrients to enter the space through the mesh lining while at least substantially preventing the marine microorganisms that are being cultivated from escaping the space through the mesh lining;
a funnel having a first opening at which the funnel is attached to a corresponding hole at the bottom of the mesh lining, and a second opening to which a hose is connected, the marine microorganisms that have been cultivated being removed from the space of the mesh lining through the funnel and then through the hose; and,
a first valve disposed through the hollow tubing of the frame and located at or near a top of the frame; and,
a second valve disposed through the hollow tubing of the frame and located at or near the bottom of the frame, the second valve operated in a substantially permanent open state in which the second valve externally exposes the interior space of the hollow tubing of the frame,
wherein the first valve has a plurality of mutually exclusive states in which the first valve is adapted to operate, the mutually exclusive states essentially consisting of:
an open state in which the first valve externally exposes the interior space of the hollow tubing of the frame, such that a level of the water within the hollow tubing of the frame rises and an extent to which the frame is submerged within the water increases;
a closed state in which the first valve at least substantially does not externally expose the interior space of the hollow tubing of the frame, such that the level of the water within the hollow tubing of the frame remains substantially constant and the extent to which the frame is submerged within the water remains substantially constant; and,
a gas-transfer state in which gas is pumped into the hollow tubing through the first valve, such that the level of the water within the hollow tubing of the frame lowers, and the extent to which the frame is submerged within the water decreases.

20. An apparatus for containing, cultivating, and harvesting photosynthetic marine microorganisms within water, comprising:
a self-supporting buoyant frame to be placed within the water;
means for permitting the water and nutrients to enter and for at least substantially preventing the marine microorganisms that are being cultivated from escaping;
means for permitting the marine microorganisms that have been cultivated to be harvested; and,
means for controlling an extent to which the frame is submerged within the water.
